(19) United States
(12) Patent Application Publication
(10) Pub. No.: US 2003/0083681 A1
(43) Pub. Date: May 1, 2003

(54) Surgical rotary abrader

(76) Inventors: Timothy E. Moutafis, Gloucester, MA (US); Brian Connor, Newfields, NH (US)

Correspondence Address: Lawrence M. Green, Wolf, Greenfield & Sacks, P.C., 600 Atlantic Avenue, Boston, MA, 02210, US

(21) Appl. No.: 10/245,820

(22) Filed: Sep. 17, 2002

Related U.S. Application Data
(60) Provisional application No. 60/322,815, filed on Sep. 17, 2001.
(60) Provisional application No. 60/322,855, filed on Sep. 17, 2001.
(60) Provisional application No. 60/322,856, filed on Sep. 17, 2001.
(60) Provisional application No. 60/322,857, filed on Sep. 17, 2001.
(60) Provisional application No. 60/322,858, filed on Sep. 17, 2001.
(60) Provisional application No. 60/380,999, filed on May 16, 2002.

(51) Int. Cl. A61B 17/32

(52) U.S. Cl. 606/167

(57) ABSTRACT

Apparatus for use as a surgical handpiece is disclosed. An exemplary apparatus includes a body, a rotatable shaft extending from the body and including a tissue contacting component such as a burr, and an outer tube connected to the body and surrounding at least a portion of the shaft. In some configurations, the minimum separation between the tissue contacting component and the outer tube is greater than the minimum separation between the shaft and the outer tube, thereby preventing the tissue contacting component from contacting the shaft upon the application of a force perpendicular to the axis of the shaft. The device may include stand-off elements positioned between the shaft and the outer tube to aid in preventing the tissue contacting component from contacting the outer tube during operation. The outer tube may be flexible with respect to the body, such as by being constructed of a relatively flexible material and/or by being flexibly connected to the body. In some embodiments, the outer tube may be more flexible than the shaft with respect to the body.

### RELATED APPLICATIONS

This non-provisional application claims the benefit under Title 35, U.S.C. §119(e) of co-pending U.S. provisional applications Serial Nos. 60/322,815, 60/322,855, 60/322,856, 60/322,857, 60/322,858, all filed Sep. 17, 2001, and U.S. provisional application Serial No. 60/380,999, filed May 16, 2002. U.S. provisional applications Nos. 60/322,815, 60/322,855, 60/322,856, 60/322,857, 60/322,858, and 60/380,999 are each incorporated herein by reference.

### FIELD OF THE INVENTION

This invention relates to instruments with rotating components for cutting, abrading, polishing, or removing tissue in a surgical procedure. Instruments according to the invention comprise a rotating component connected to a rotatable shaft at least partially enclosed by a sheath.

### BACKGROUND

Tissue contacting components, such as burrs, cutters, abraders, and polishers (referred to collectively herein as “burrs”) that are driven by rotating shafts are known. In the case of an open surgical field, uncovered, power driven burrs may be used because, typically, no sheath is required to protect adjacent tissue from the rotating burr or shaft. In endoscopic surgery, however, and in other surgery in tight or confined spaces in the body, it is typically important to provide a sheath or a similar device to shield the burr and, in some cases, the shaft, from contact with the tissue, so that tissue near the intended operative site is not inadvertently removed or damaged. When operating on soft tissue, little force applied to the tissue by the burr is typically needed to effect tissue removal, and movement of the burr towards the sheath, for example by bending of the burr's drive shaft, has not been appreciated as being a concern. When a burr is used on harder tissue such as bone or cartilage, however, the levels of lateral forces (i.e., forces in a direction perpendicular to the longitudinal axis of the shaft) that need to be applied to the burr can force the burr against the sheath in many prior art handpiece configurations. This can potentially damage the burr, as well as the sheath, and can create undesirable shavings or fragments of one or both.

Prior art attempts to address this problem, while potentially suitable at moderate speeds of operation, for example 100 to 8,000 revolutions per minute (“rpm”), are not well suited for use with high-speed burrs, e.g. those operating in excess of the above range, such as those operating at tens of thousands of rpm. One problem is that simple slip bearings which are positioned between the shaft and the sheath and used as support elements in some prior art designs can cause substantial friction at higher speeds, while known support elements resulting in lower friction are prohibitively expensive.

### SUMMARY OF THE INVENTION

We have found that the above-described and other difficulties can be circumvented or mitigated by configuring a surgical instrument with a rotating component as provided according to certain embodiments of the present invention. Certain embodiments of the instruments according to the invention can operate at high speeds on hard tissue without undesirable contact between the burr and the shaft of the instrument, yet can have fewer parts than typical prior art designs.

In one aspect, the invention involves instruments for use in surgery. In one embodiment, the invention comprises a body having a distal end and a proximal end, a shaft rotatably supported by the body and extending from the distal end of the body, a tissue contacting component drivable by the shaft, and an outer tube connected to the distal end of the body and surrounding at least a portion of the shaft, wherein a minimum separation between the tissue contacting component and the outer tube is greater than a minimum separation between the shaft and the outer tube.

In another embodiment, the invention comprises a body having a distal end and a proximal end, a shaft rotatably supported by the body and extending from the distal end of the body, a tissue contacting component drivable by the shaft, and an outer tube surrounding at least a portion of the shaft, wherein the outer tube is constructed and arranged such that, upon application of lateral force to the tissue contacting component, the outer tube contacts the shaft before contacting the tissue contacting component.

In yet another embodiment, the invention comprises a body having a distal end and a proximal end, a shaft rotatably supported by the body and extending from the distal end of the body, a tissue contacting component drivable by the shaft, an outer tube flexibly connected to the distal end of the body and surrounding at least a portion of the shaft, and at least one stand-off constructed and arranged such that, upon application of a lateral force to the tissue contacting component, the at least one standoff contacts the shaft before the tissue contacting component contacts the outer tube.

Another embodiment of the invention comprises a body having a distal end and a proximal end, a shaft rotatably supported by the body and extending from the distal end of the body, a tissue contacting component drivable by the shaft, a flexible connector member positioned at the distal end of the body, and an outer tube connected to the flexible connector member and surrounding at least a portion of the shaft.

In another embodiment, the invention comprises a body having a distal end and a proximal end, a shaft rotatably supported by the body and extending from the distal end of the body, a tissue contacting component drivable by the shaft, and an outer tube connected to the distal end of the body and surrounding at least a portion of the shaft, wherein the outer tube is more flexible than the shaft.

In another aspect, the invention involves shaft assemblies for use in surgical instruments. One embodiment of this aspect of the invention comprises a rotatable shaft, a tissue contacting component drivable by the shaft, and an outer tube surrounding at least a portion of the shaft, wherein a minimum separation between the tissue contacting component and the outer tube is greater than a minimum separation between the shaft and the outer tube.

In another aspect, the invention involves a method. One embodiment of this aspect of the invention comprises providing a surgical instrument including a body having a distal end and a proximal end, a shaft roatably supported by the body and extending from the distal end of the body, a tissue contacting component drivable by the shaft, and an outer tube positioned to surround at least a portion of the shaft and tissue contacting component and having an inner surface normally radially spaced from an outer surface of the shaft; contacting the tissue contacting component with tissue of a patient; applying a force to the tissue via the shaft and tissue contacting component, at least a portion the force being laterally directed with respect to the shaft; and laterally displacing at least a portion of the shaft with respect to the outer tube in response to application of the force, thereby decreasing a radial spacing between the outer surface of the shaft and the inner surface of the outer tube without contact between the inner surface of the outer tube and the tissue contacting component.

In another embodiment of this aspect of the invention method comprises providing a surgical instrument including a body having a distal end and a proximal end, a shaft roatably supported by the body and extending from the distal end of the body, a tissue contacting component drivable by the shaft, an outer tube positioned to surround at least a portion of the shaft and tissue contacting component and having an inner surface normally radially spaced from an outer surface of the shaft, and at least one stand off positioned between the shaft and the outer tube; contacting the tissue contacting component with tissue of a patient; applying a force to the tissue via the shaft and tissue contacting component, at least a portion the force being laterally directed with respect to the shaft; and laterally displacing at least a portion of the shaft with respect to the outer tube in response to application of the force, thereby decreasing a radial spacing between the outer surface of the shaft and the inner surface of the outer tube without contact between the inner surface of the outer tube and the tissue contacting component.

### BRIEF DESCRIPTION OF THE DRAWINGS

Other advantages, novel features, and uses of the invention will become more apparent from the following detailed description of the invention when considered in conjunction with the accompanying drawings, in which:

FIG. 1 is a plan view of a surgical instrument according to one aspect of the invention;

FIG. 2a is a cross-sectional view of the instrument of FIG. 1, taken along line A-A;

FIG. 2b is a detailed cross-section of the distal end of the device of FIG. 1;

FIG. 2c is a detailed cross-section of the proximal end of the device of FIG. 1;

FIG. 3a is an enlarged view of the distal end of another embodiment of the instrument illustrated in FIG. 1;

FIG. 3b is an enlarged view of the distal end of another embodiment of the instrument illustrated in FIG. 1;

FIG. 3c is an enlarged view of the distal end of another embodiment of the instrument illustrated in FIG. 1;

FIG. 3d is an enlarged cross-sectional view of the distal end of another embodiment of the instrument illustrated in FIG. 1;

FIG. 4 is a partially cut away perspective view of the instrument of FIG. 1;

FIG. 5 is a cross-sectional view of a portion of an alternative embodiment of a surgical instrument according to one aspect of the invention;

FIG. 6a is a cross-sectional view of an alternative embodiment of a surgical instrument according to the invention; and

FIG. 6b is a perspective view of a cross-section of an alternative embodiment of a surgical instrument according to the invention.

The drawings are schematic and are not intended to be drawn to scale. In the figures, each identical or substantially similar component that is illustrated in various figures is typically represented by a single numeral or notation. For purposes of clarity, not every component is labeled in every figure, nor is every component of each embodiment of the invention shown where illustration is not necessary to allow those of ordinary skill in the art to understand the invention.

### DETAILED DESCRIPTION

Exemplary embodiments of the invention provide surgical instruments with rotating components suitable for high speed operation, which can also, in some embodiments, be simpler to manufacture than many typical prior art instruments. In one aspect of the invention, a surgical instrument with a burr connected to a rotating shaft has a minimum separation between the burr and a surrounding sheath (also referred to herein and in the claims as an “outer tube”) that is greater than a minimum separation between the shaft and a portion of the sheath that surrounds the shaft. The term “minimum separation,” as it is used herein and in the claims, refers to the smallest radial distance between the sheath and the burr, in the former instance, and between the sheath and the shaft, in the later instance, at any longitudinal position along the length of the shaft and the burr. In some embodiments, the space between the outside diameter of the shaft and the inside diameter of the sheath creates a channel along at least of portion of the sheath through which fluid and/or debris can flow.

In one embodiment, for example, the burr may be smaller in diameter than the shaft, while the sheath diameter may be at least slightly larger than the diameter of the shaft and approximately constant along its length. Upon lateral deflection of the shaft relative to the sheath, such as when the shaft is subject to a load created when the burr is being used to cut or abrade hard or dense tissue or bone, contact between the burr and the sheath will be avoided because the relative lateral movement of the shaft (and, thus, the burr) will be arrested by contact between the outer surface of the shaft and the inner surface of the sheath, which will occur before the burr touches the sheath.

In alternative embodiments, the diameter of the burr can be approximately the same as the diameter of the shaft, or even larger than the diameter of the shaft, and the inner diameter of the sheath can be at least slightly larger than the diameter of the shaft in a region proximal to the burr (i.e., in a region surrounding the shaft), but flared outwards in the region surrounding the burr in an amount sufficient to provide the desired relative clearance with the burr. As with the above-described embodiment, relative lateral movement of the shaft will be arrested by the sheath prior to contact between the burr and the sheath. In some embodiments, the flaring may be a characteristic of the inner diameter of the sheath only, i.e., the outer diameter of the sheath may be substantially constant while the inner diameter of the sheath may increase in the area of the burr. The substantially uniform outside diameter provided by such an arrangement may facilitate easier insertion and removal of the shaft from an incision. Of course, the outer diameter of the sheath need not be constant and may instead have any suitable shape or contour. The precise shape of the flare of the internal/external diameter of the sheath in the region of the burr may also be tailored to a particular burr shape, so as to, for example, provide as low a profile as practicable or maintain a substantially uniform cross-sectional area of any channel formed between the shaft and the sheath.

Because contact between the burr and the sheath may be avoided in the above constructions without use of a bearing positioned in the distal region of the shaft, construction and manufacture of a rotating surgical instrument according to the invention can potentially be simplified over that of typical prior art instruments.

In some embodiments, provision of the relative minimum separations described above may be acheived by providing one or more “stand-offs” positioned in the space between the shaft and the sheath. The term “stand-off,” as used herein and in the claims, refers to any structure that allows the shaft to float freely within the sheath (i.e., without direct or indirect contact between the shaft and the sheath) but is capable of maintaining a fixed distance between the shaft and the sheath upon the application of lateral force, while still allowing the shaft to be displaced laterally upon the application of lateral force. Suitable structures include, for example, ribs, feet, fins, rings, pins, knobs, ridges, buttons, or any other appropriate element or arrangement. Stand-offs may be formed integrally with the shaft and/or the sheath, may be separate structures affixed to the shaft and/or the sheath by any suitable means, or may simply be positioned in the space between the shaft and the sheath. In some embodiments, stand-offs may be formed or positioned on both the sheath and the shaft and directly adjacent to one another, such that the minimum separation lies between the stand-offs.

It should be understood that stand-offs are an optional feature and that, accordingly, many embodiments are entirely free of stand-offs. Indeed, in at least one embodiment, it is an advantageous feature of the invention that the region of the sheath and shaft distal of the body is devoid of stand-offs or any other element that might impede the flow of fluid though the sheath or that might complicate the construction and/or manufacture of the instrument.

In another aspect of the invention, a surgical instrument is provided with a flexible sheath that surrounds at least a portion of a rotating shaft and, optionally, a portion of the burr as well. During operation of such instrument, the shaft may contact the sheath, but because the sheath is flexible and displacable in response to the contact, rotation of the shaft is subject to less frictional resistance than would be the case with a relatively rigid sheath. “Flexible” is used herein and in the claims to mean that the sheath itself and/or the connection between the sheath and a body of the instrument is compliant and can swivel or bend to allow the sheath to comply, at least partially, with bending or lateral displacement experienced by the shaft.

In some embodiments, the sheath (e.g., a sheath formed of a substantially rigid material) may be rendered “flexible” by reason of being affixed to the body by a connection herein referred to as a “flexible connection.” A variety of types of flexible connections are contemplated, including, without limitation, types with elastomeric, resilient, and/or reversibly bendable elements, types with a swivelable and/or pivotable joint, types in which the fit between the sheath and a projecting hollow member has a large clearance, and/or even hinge-type connectors. The flexible connection between the sheath and the body in some embodiments could also be formed by corrugated or non-corrugated sections of tubing, made of essentially any resilient, medically-compatible material, linking the sheath and the body. In other embodiments, the sheath could be loosely connected to the body by a mechanical arrangement, such as a ball and socket joint or similar device, that would allow bending and/or pivoting without requiring elastic elements. Any other means for providing attachment of a sheath (e.g., one formed of a substantially rigid tube) to a body, or more particularly to a body of a surgical instrument, in a way that allows bending or pivoting of the sheath with respect to the axis of the body during operation of the instrument may also be used.

In one embodiment, the flexible connection takes the form of a boot formed of a resilient material, such as rubber or soft plastic, that fits into an annular recess in the distal end of the body. The boot can have a central bore that surrounds the shaft and a portion of the sheath and an annular recess in the bore that accepts a flared proximal end of the sheath. The flexibility of the material that forms the boot can allow the sheath to flex upon application of force with a component in a direction perpendicular to the axis of the shaft. This is but one arrangement, however, and, as described above, the invention contemplates the use of any other suitable type of flexible connection.

Instead of, or in addition to, having a flexible connection between the sheath and the body of the handpiece, some embodiments of the invention may alternatively provide a sheath at least of portion of which is flexible by reason of being composed of a resilient, supple, or pliable material and that has dimensions (e.g., thickness, diameter, etc.) that allow it to flex or bend along its length in response to the application of a lateral force. Such bending or flexing can result in less resistance to the bending and/or lateral displacement of the shaft. The flexibility of the sheath may be substantially uniform or non-uniform along its length; for example, the sheath may be more flexible in its proximal portions, so as to facilitate bending, while being less flexible in its distal portions, so as to resist external forces that might operate to push the sheath in the direction of the burr. The non-uniform flexibility may, in some cases, result from the use of a sheath that is substantially rigid but connected to the body via a flexible connector.

In one embodiment, the surgical instrument is constructed such that the sheath is more flexible than the shaft, meaning that the level of force required at a given longitudinal point to deflect the sheath a given distance is less than the level of force required at the same longitudinal point to deflect the shaft the same distance. In one embodiment, the level of force at a given longitudinal point required to deflect the sheath a given distance is less than about 90% of that required to deflect the shaft the same amount. In other embodiments, the level of force required to deflect the sheath may be less than about 75%, 50%, 25%, 10%, 5%, 2% or 1% of the force required to deflect the shaft.

It should be noted that, regardless of the means by which the sheath is made flexible, the displacement along the entire length of the sheath during operation will typically be small, in the range of ten degrees or less, and, more typically, a few degrees or less.

In some embodiments, a combination of the above-described features is provided; that is, the sheath has a greater clearance from the burr than it does from the shaft, and the sheath is flexible and/or is flexibly connected to the body. Such an embodiment is well suited to prevent contact between the sheath and the burr, as the clearance and the flexing of the shaft and/or the flexible connection cooperate to help maintain a separation between the sheath and the burr.

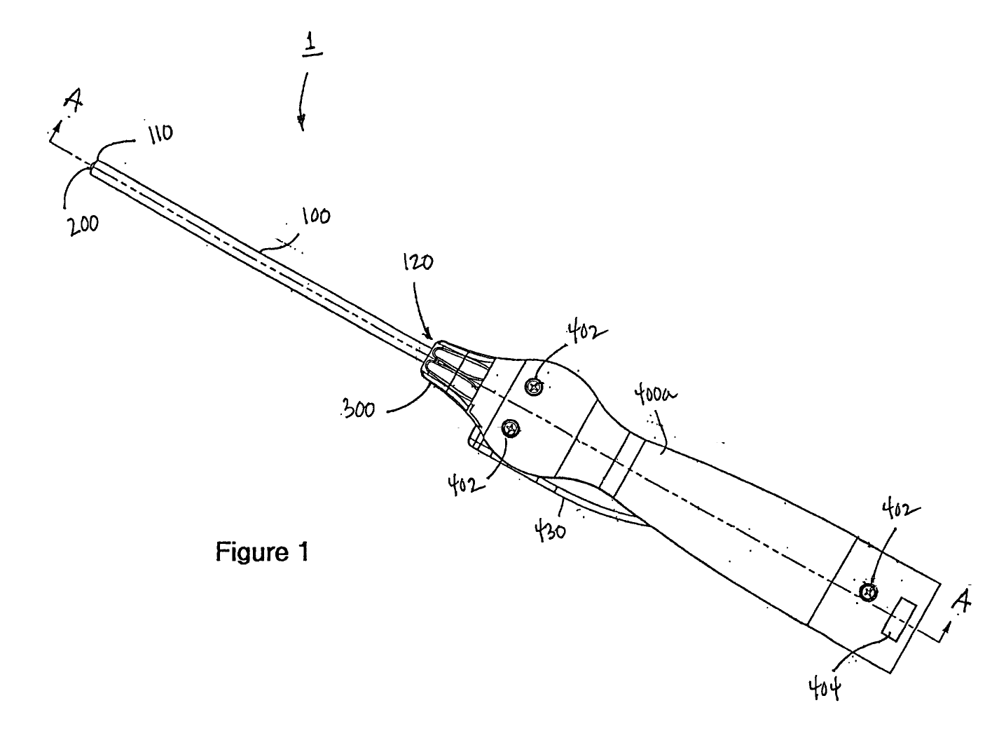

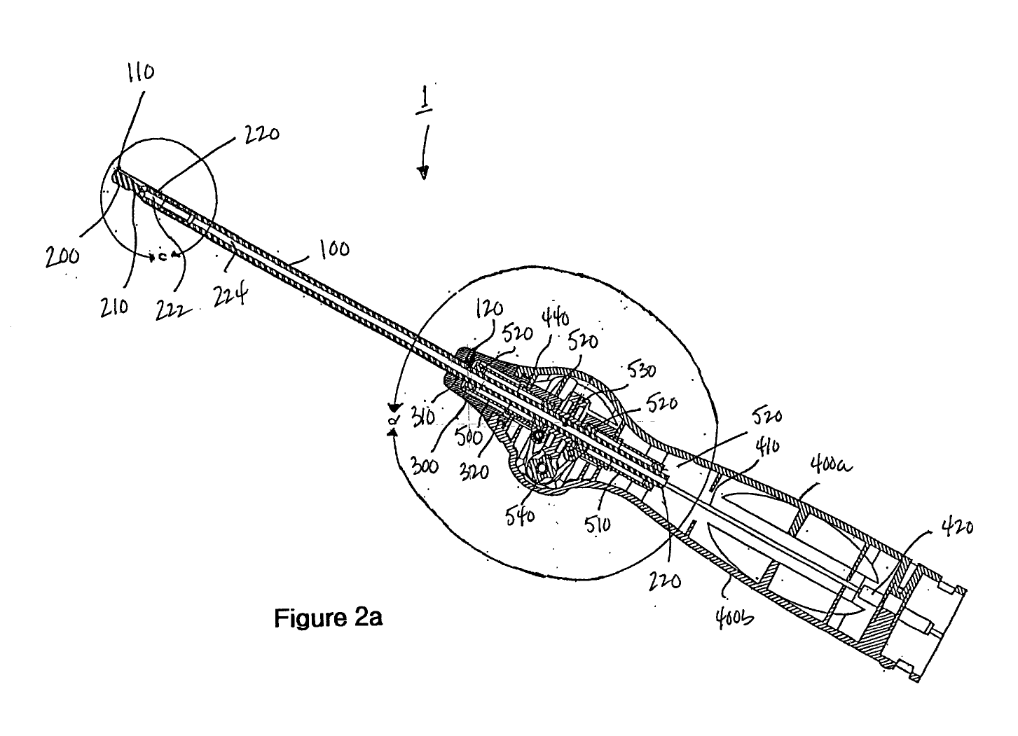

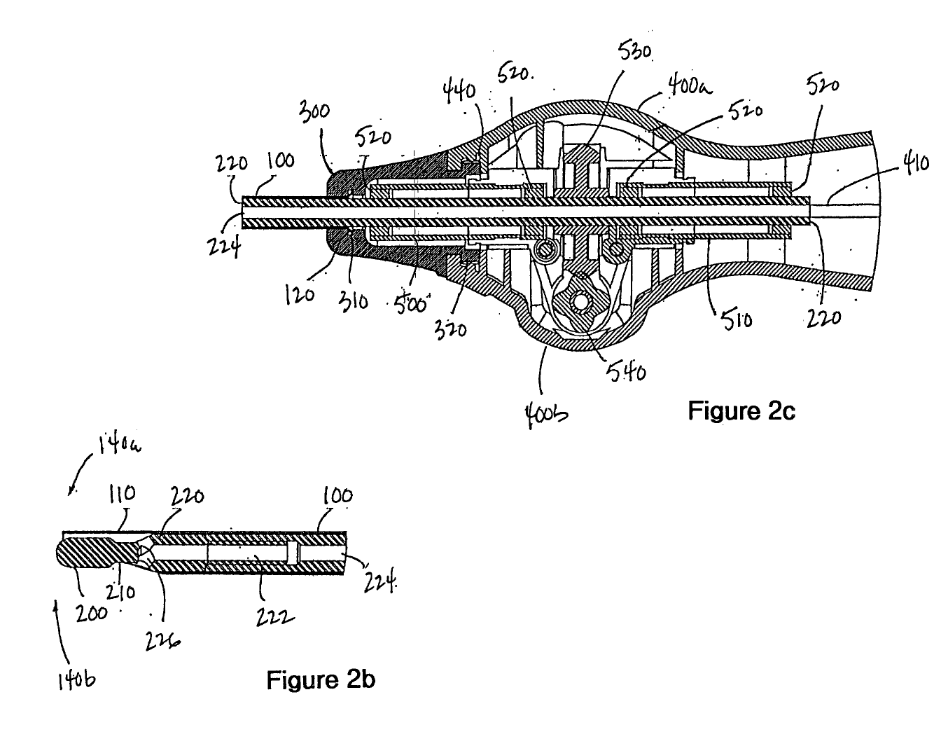

An embodiment of the invention is now described with reference to the figures to aid the experienced person in understanding certain aspects of the invention and in envisaging how it may be practiced. FIGS. 1 and 2 show one exemplary surgical instrument according to the invention. As shown in FIG. 1, the exemplary device 1 includes a sheath 100 with a distal tip 110 and a proximal end 120, a shaft 220 (see FIG. 2a), and a burr 200.

FIG. 2a, and enlargements 2b and 2c, show cross-sectional views of the surgical instrument of FIG. 1. FIG. 2b shows an enlarged view of the burr 200, shaft 220, and sheath 100. The burr 200 of this embodiment is manufactured in a single piece with neck 210 and shaft 220. In other embodiments, however, the burr 200, neck 210, and shaft 220 may be assembled after manufacture, either reversibly or irreversibly. Suitable methods of joining these components include the use of a tang that is optionally hidden after the connection is made, direct welding, threads, a press fit, or any other appropriate method for joining cylindrical objects end-to-end. In other embodiments, the neck 210 may be diminished, or omitted entirely, so that the burr 200 is directly connected to the shaft 220. The burr 200 of this embodiment has a diameter that is smaller than that of the shaft 220. As noted above, however, in other embodiments, the burr 200 may be of the same or a larger diameter than the shaft 220.

While this embodiment includes a burr 200 with a shape particularly suitable for abrading tissue, it should be understood that any type of abrasive, cutting, polishing, or other tissue contacting element is potentially suitable for use in place of the burr 200 according to the invention. The teeth of the burr 200, for instruments including burrs with teeth, may be cut into a body, as is conventional, or may be provided in the form of raised ridges of various profiles. Such profiles can be effective, even for fairly smooth profiles, due to the high rotational speeds achievable by some embodiments of instruments provided according to the invention. In some embodiments, a burr may be provided that comprises lateral linear projections from a body portion, such as wires or polymeric bristles, which may be flexible or rigid, so that the burr can act as a brush that, for example, can scour tissue to which it is applied. In other, related embodiments, a burr may be utilized that is analogous in structure and function to a rotary string trimmer/cutter. In some such embodiments, the burr can comprise one or more components including a feed mechanism for supplying one or more cutting filaments formed, for example, of polymer, metal wire, etc., in a cutting/trimming configuration. Such feed mechanism can, in some such embodiments, be configured to reversibly feed such filaments through apertures in the feed mechanism, such that abrasion, trimming, and/or cutting can be achieved upon rotation of a shaft of the instrument to which such feed mechanism is attached. In certain of such embodiments, the feed mechanism can comprise a head, which includes the apertures and contains the filaments, carried on the shaft.

The burr 200 may be made of a material that can keep a sharp edge and that will not react chemically with the tissue. Metals, including steel, more especially stainless steel, are most commonly used for such purposes, and often the steel or other metal will be hardened. Ceramic cutters, or cutters coated with hard ceramic particles, diamond dust, other abrasives, or grit are also known. In very high speed burrs, some plastics may provide sufficient cutting action for soft tissue, or may be suitable for polishing.

The shaft 220 of this embodiment is approximately cylindrical has a central lumen 224. In other embodiments, however, the shaft 220 may be wholly or partially solid. The length and diameter of the shaft 220 may vary depending on the application although, in certain embodiments, the diameter is approximately constant along its length. The shaft 220 can be made of stainless steel, but, alternatively, it may be made of other materials having sufficient strength, including, without limitation, metals, and particularly metals selected from other steels, aluminum, titanium and bronze. Those of ordinary skill in the art will, based on well-known material property data and no more than routine experimentation, readily be able to evaluate candidate materials or combinations of materials and determine if their properties are appropriate.

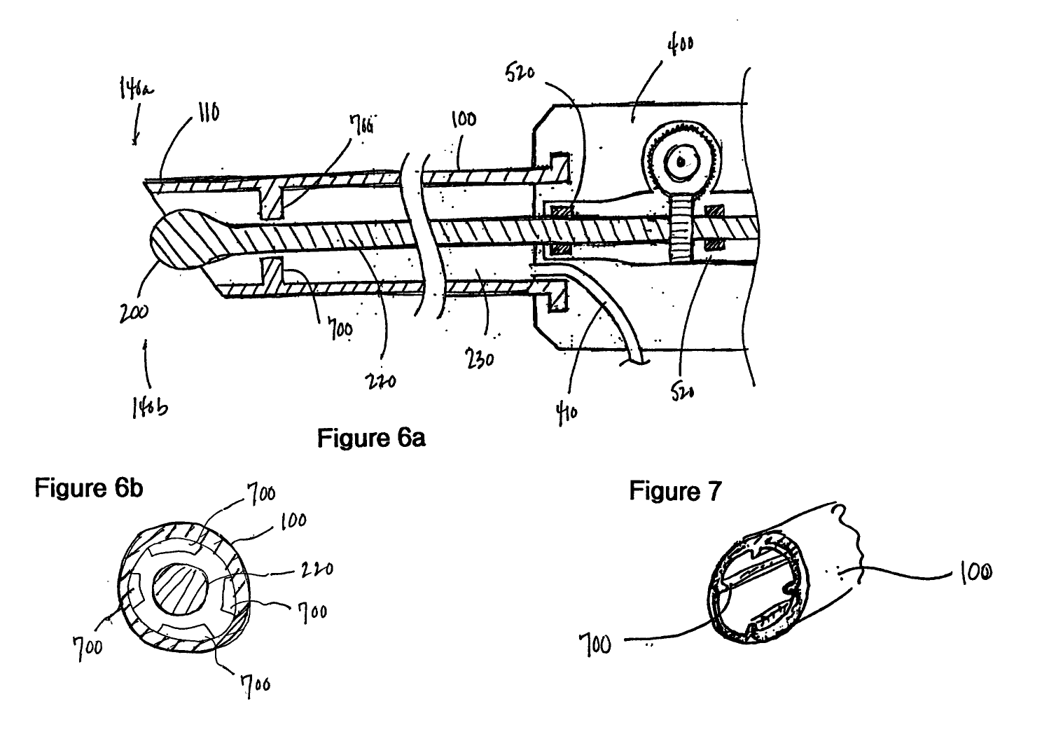

In the illustrated embodiment, the shaft 220 and burr 200 are both at least partially surrounded by sheath 100. There is separation between the shaft 220 and the sheath 100 which forms a channel 230 (shown in FIG. 6a). In this embodiment, the minimum separation between the burr 200 and sheath 100 is greater than the minimum separation between the shaft 220 and the sheath 100. As a result of the difference in these minimum separations, upon the application of a lateral force to the burr 200, the shaft 220 may deflect relative to the sheath 100 until the shaft contacts the sheath 100. Once the shaft 220 contacts the sheath 100, the relative deflection will stop and the burr 200 will be prevented from contacting the sheath 100.

In some alternative embodiments (not shown), the distance between the sheath and the shaft may not be constant along the length of the shaft. For example, the inner diameter of the sheath may be larger than the outside diameter of the shaft by a certain amount for some distance from the proximal end of the shaft and then may have a region in which the difference in the diameters is smaller, forming a neck in a mid-region of the shaft that is the location of the minimum separation, and then may widen in the region distal and/or proximal to the neck.

In the illustrated embodiments, the sheath 100 is configured as a tube, which may be made of a material that is more easily displaced laterally than the shaft, but that has a sufficient stiffness to resist significant bending deformation by the lateral stresses likely to be encountered in ordinary use. In particular, the sheath 100 may be constructed to be rigid enough to prevent it from coming into contact with the burr 200, while being sufficiently movable with respect to the body that it can, under expected operating conditions, flex, bend, or pivot along its length in response to the application of a lateral force. The bending, flexing, or pivoting may result in less resistance to the bending of the shaft 220. As noted above, the flexibility of the sheath 100 can also vary, in some embodiments, along its length, and such variable flexibility may be effected, for example, by varying the composition of the material that forms the sheath 100, by varying the thickness of all or part of the sheath 100, or by the inclusion of a flexible connector.

The sheath 100 of some embodiments of the invention, such as that illustrated in FIGS. 1 and 2, may also function to prevent contact between the shaft 220 and adjacent tissue. Some such embodiments may provide a sheath 100 configured such that the sheath covers all or part of the shaft 220, an arrangement that can prevent damage to adjacent tissue that might result from friction or from imperfections, such as scratches, on the shaft 220. In some embodiments, and as illustrated in FIGS. 1 and 2, the sheath 100 may fully encircle the shaft, so as to protect tissue from contact with the rotating shaft 220 around its entire circumference. In other embodiments, the sheath may cover only a portion of the shaft, leaving other portions of the shaft exposed. In still further embodiments, the sheath may have any of a variety of perforations, in the form of holes, slots, slits, or the like to, for example, reduce the weight of the instrument or to facilitate various fluid flow arrangements, while still providing sufficient strength and/or shielding.

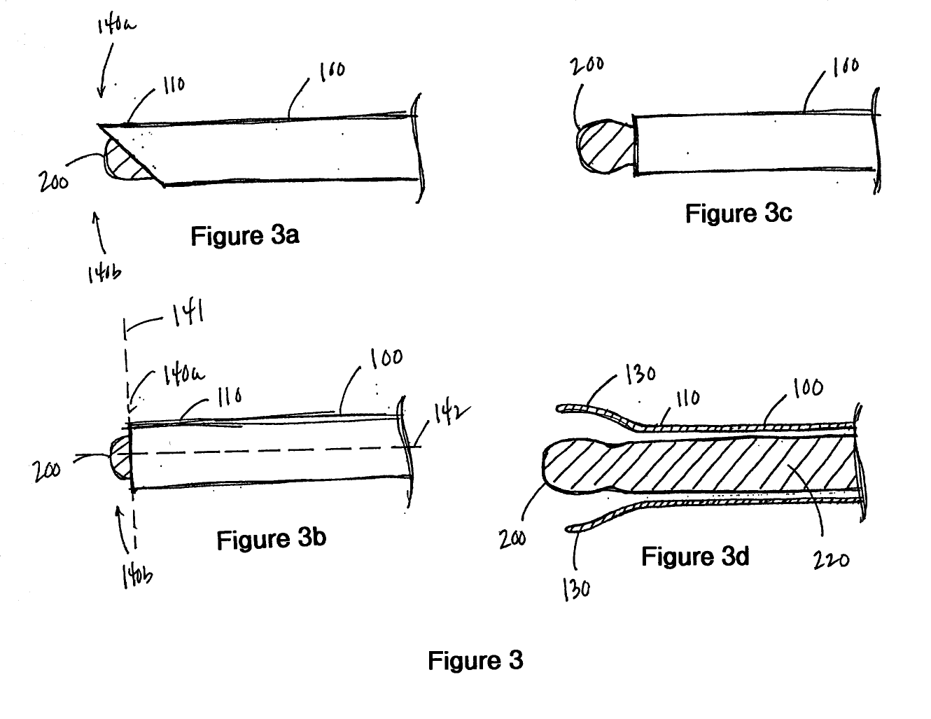

In certain embodiments, the sheath may surround only a portion, or even variable portions, of the burr. In some embodiments, for example, the sheath may cover only a portion of the burr. Such an arrangement is shown in FIG. 2b, where the sheath 100 extends nearly to the distal end of the burr 200 on the top side 140a of the distal end 110, but is cut away, such that the distal end of the sheath 100 forms an acute angle with the longitudinal axis of the shaft 220, to reveal a large portion of the burr 200 on the bottom side 140b of the distal end 110. This type of construction provides protection to tissues located on the top side 140a, while allowing the burr 200 to contact tissue located on the bottom side 140b. A non-sectional view of a similar arrangement is shown in FIG. 3a. Alternatively, the distal end 110 of the sheath 100 may terminate in a plane 141 perpendicular to the longitudinal axis 142 of the shaft 220 and passing through the burr 200, as shown in FIG. 3b, in which case a larger or smaller portion of the burr 200 may be exposed, depending on where the distal end of the sheath 100 stops in relation to the burr 200. The sheath 100 may also be constructed and positioned to expose substantially all of the burr 200, as shown in FIG. 3c.

In the embodiment illustrated in cross-section in FIG. 3d, a portion of the sheath 100 is expanded in diameter at its distal end to form, for example, a hood 130, so as to increase the separation between the inner surface of the hood 130 and the burr 200. The sheath 100 may also have any of a number of other configurations, as would be apparent to one of skill in the art, that would expose only selected portions of the burr 200.

In still other embodiments, the sheath may be constructed and arranged so that the point at which the distal end of the sheath terminates may be variable, allowing the amount of the burr that is exposed to be changed. Such variable exposure may be effected, for example, by a bellows-type arrangement, a slidable or threaded sheath, a telescoping sheath arrangement, or any other suitable method, as would be appreciated by those of skill in the art. In some such embodiments, the exposure of the burr may be adjusted during the course of a procedure, either manually or by some type of automatic control.

In general, the sheath 100 may have any suitable thickness and external diameter and may be constructed of any appropriate material, selection of which dimensions and materials is well within the abilities of one of skill in the art given the guidance and teaching of the present description. In one exemplary embodiment, the sheath 100 is made of stainless steel, but, alternatively, it may be made of other materials having sufficient strength, including without limitation metals, and particularly metals selected from other steels, aluminum, titanium, bronze, and copper and its alloys. In some embodiments, the sheath 100 may be made of rigid plastic, although the material should preferably be non-melting under expected operating conditions. In some embodiments, the sheath 100 may be optically transparent to aid visualization of the burr 200 and/or may be provided with a radiopaque portion or element to further facilitate visualization. In still other embodiments, the sheath 100 may be made of a combination of one or more of these materials. In still other embodiments, the sheath may be made of a substantially resilient, pliant, and/or flexible material, as previously discussed. As with the shaft 200, those of ordinary skill in the art will, based on well-known material property data and routine experimentation, readily be able to evaluate candidate materials to see if they possess appropriate properties. The material of the sheath 100 may be selected in certain embodiments from a material that does not readily gall on contact with the burr 200.

For embodiments in which removal of fluid or debris from the surgical site is desired, it can be effected by providing for removal through a lumen in the shaft and/or by providing a channel between the shaft and the sheath. In embodiments in which the shaft has a lumen, the shaft may be provided with openings, which may, in some embodiments, be positioned near its distal end, to allow fluid and/or debris to flow into the lumen. The embodiment of FIGS. 1 and 2, for example, includes a hollow shaft 220 providing a lumen 224 with inlets 226 that fluidly connect the area surrounding the burr 200 with the entry lumen 222. When used, inlets 226 may, in certain embodiments, be arranged symmetrically around the shaft 220, for balance during rotation. As shown in FIG. 2c, the entry lumen 222 is fluidly connected to shaft lumen 224, and shaft lumen 224 is in fluid communication with a proximal tube 410 that extends from the proximal end of the shaft 220 and passes through a carrier block 420 (see FIG. 2a) to the proximal end of the body. Providing for the evacuation of fluid through a channel 230 between the shaft 220 and the sheath 100 is particularly suitable for embodiments of the invention in which the region of the sheath 100 and shaft 220 distal of the body is free of any support member or other element tending to obstruct channel 230. In typical embodiments, the suction necessary to induce and maintain evacuation flow through the lumen 224 and/or the channel 230 can be provided, for example, by elevation of a bag of saline used for irrigating the surgical site or by the design of the burr to act as an impeller as it rotates. In some embodiments, it is contemplated that fluid could be delivered to or evacuated from the site through either or both of a shaft lumen or a channel between the shaft and the sheath, either simultaneous or sequentially.

Some embodiments may also include a flexible connector positioned between the sheath and the body. In the embodiment shown in FIG. 2c, for example, the sheath 100 passes into the connector 300 and the flared proximal end 120 of the sheath 100 is held in an annular groove 310. The connector 300 of this embodiment is made of a flexible, resilient material, such as a rubber. In other embodiments, the connector 300 may take other shapes than illustrated and/or may be made of other flexible materials, such as, for example, other resilient polymeric materials and certain metals. As illustrated, connector 300 has a rim 320 that fits into a slot 440 in body 400. Because the connector 300 is flexible, application of lateral force to the sheath 100 or the burr 200, for example via contact with the shaft 220 during operation, will tend to cause the connector 300 to flex in the direction of the applied force, which will cause the sheath 100 to pivot in that direction with respect to the flexible connector 300, thereby reducing the possibility that the burr 200 might contact the sheath 100 and reducing the friction between the shaft 220 and the sheath 100. In alternative embodiments, the rubber flexible connector 300 may be replaced by, and/or supplemented with, a connector made of other types of elastomeric, resilient, and/or reversibly bendable materials. In other alternative embodiments, the illustrated flexible connector may be replaced by and/or supplemented with a pivotable mechanical connection, such as a swivelable and/or pivotable joint, a hinge, corrugated sections of tubing, a ball and socket joint or other similar device, that would allow bending and/or pivoting with or without requiring elastic elements. In essence, essentially any suitable means for providing attachment of a tube to a body of a surgical instrument in a way that would allow relatively easy bending or pivoting of the tube with respect to the axis of the body can potentially be employed within the scope of the invention.

Surgical device 1 illustrates an embodiment that includes an optional body 400. While the body 400 of the illustrated embodiment is formed of two injection molded plastic sides 400a, 400b, in other embodiments the body 400 may be formed of any material suitable for use in a surgical instrument including, without limitation, any appropriate plastic or metal and may be formed of any suitable number of parts, including one. Where the body 400 is formed from more than one interconnected piece, the pieces may be held together by any suitable means. In the embodiment of FIGS. 1 and 2, for example, screws 402 are illustrated. In other embodiments, however, the screws 402 could be replaced by rivets, clamps, adhesives, a snap fit, or any other appropriate method of fastening the parts of the body together. The exterior of the body 400 may also be sized and shaped to be held in a hand, as shown, and may be adapted to reversibly or irreversibly mate with a device (not shown) that provides feed lines for fluid influx and efflux. In the illustrated embodiment, a locking slot 404 at the distal end of the body accommodates a tab (also not shown) for attaching the body 400 to such a device.

In some embodiments, as illustrated in FIGS. 1 and 2, the body 400 also houses support members configured and positioned for rotatably supporting the shaft. Such support members may take the form of, for example, roller or non-roller bearings, bushings, o-rings, washers, ribs, feet, fins, rings, pins, knobs, ridges, buttons, or any other appropriate element or arrangement, as would be apparent to one of skill in the art. Within the body 400 of the illustrated embodiment, for example, a first support tube 500 and a second support tube 510 each carry two support members 520 that rotatably support the shaft 220. The support members 520 may fully encircle the shaft 220 or may be discontinuous around the circumference of the shaft, so long as they are constructed positioned so as to provide appropriate rotational support. In some embodiments, the support members 520 may simply be portions (e.g. molded portions) of the body of the body itself which are constructed and arranged to rotatably support the shaft.

In the embodiment of FIGS. 1 and 2, the shaft 220 is rotatably supported solely by support members 520, each of which are positioned within the body 400, thus resulting in a cantilevered arrangement. Such an arrangement can provide sufficient lateral support of the shaft 220 to allow the device to be used on hard tissue without the burr 200 contacting the sheath, particularly when used in conjunction with the minimum separation, flexible sheath, and/or flexible connector arrangements described above for preventing burr-shaft contact. This type of support member arrangement can also facilitate a construction, such as that illustrated, in which the region of the sheath 100 and shaft 220 distal of the body 400 is devoid of any bearings, support members, or elements that might block the flow of fluid though the space between the sheath 100 and shaft 220 and/or complicate the construction and/or manufacture of the instrument. In other embodiments other than those illustrated, the support members may be positioned wholly or partially outside of the body, on either its distal or its proximal side.

As illustrated in FIGS. 1 and 2, the shaft 220 may be drivingly connected and/or affixed to a driving element (also referred to herein and in the claims as a “motor”), which may be any device or arrangement capable of imparting rotation to the shaft. The motor of the illustrated embodiment is a liquid-jet driven rotor drive mechanism similar to that described in commonly owned co-pending U.S. patent application Ser. No. 09/480,500 and International Publication No. WO 01/50966, both incorporated herein by reference. This drive mechanism can deliver both high speed and high torque, and tends to slow and stall smoothly as torque increases. In alternative embodiments, however, other drive mechanisms may be utilized in the invention. In particular, an air turbine is may be used in certain embodiments, as may an electric motor.

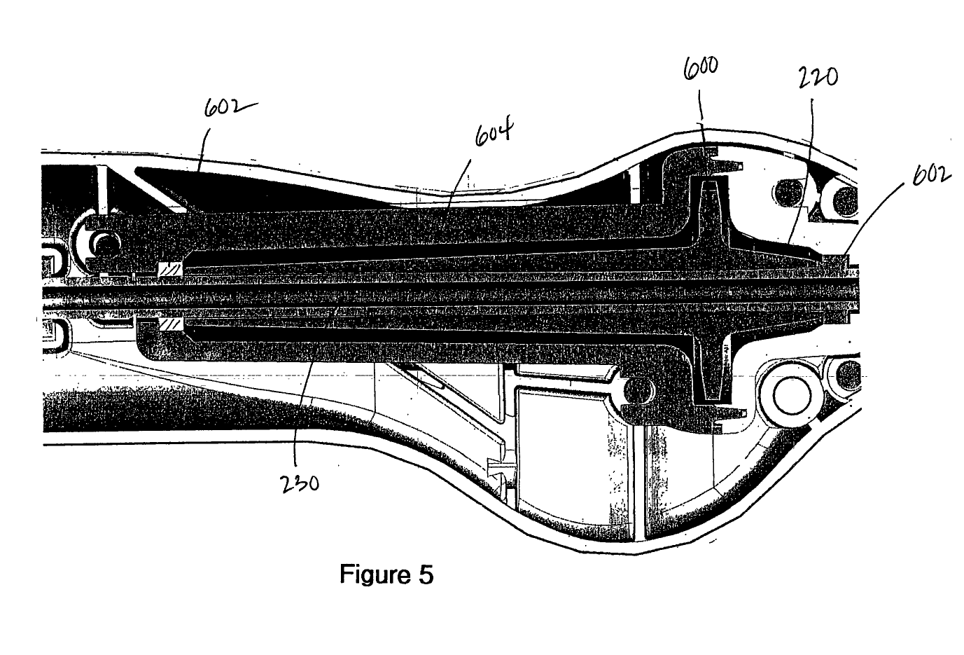

In the embodiment illustrated in FIGS. 1, 2, and 4, a driving gear 530 connects the shaft 220 to the liquid jet-driven rotor 550. The gear 530 can be made of any suitable material, including but not limited to metal and plastic, may be any suitable shape, and may be affixed to the shaft by any suitable means, as would be apparent to those skilled in the art. In other embodiments, the shaft 220 and the gear 530 may be formed from a single piece of material. FIG. 5, for example, depicts an alternative embodiment in which the water-jet driven rotor 600 is coupled directly to the shaft 220. The shaft 220 is supported by two support members 602, and encased by a support member 604, which combines the functions of the first 500 and second 510 support tubes, and the connecting block 12 of the previous embodiment, illustrated in FIGS. 1, 2, and 4.

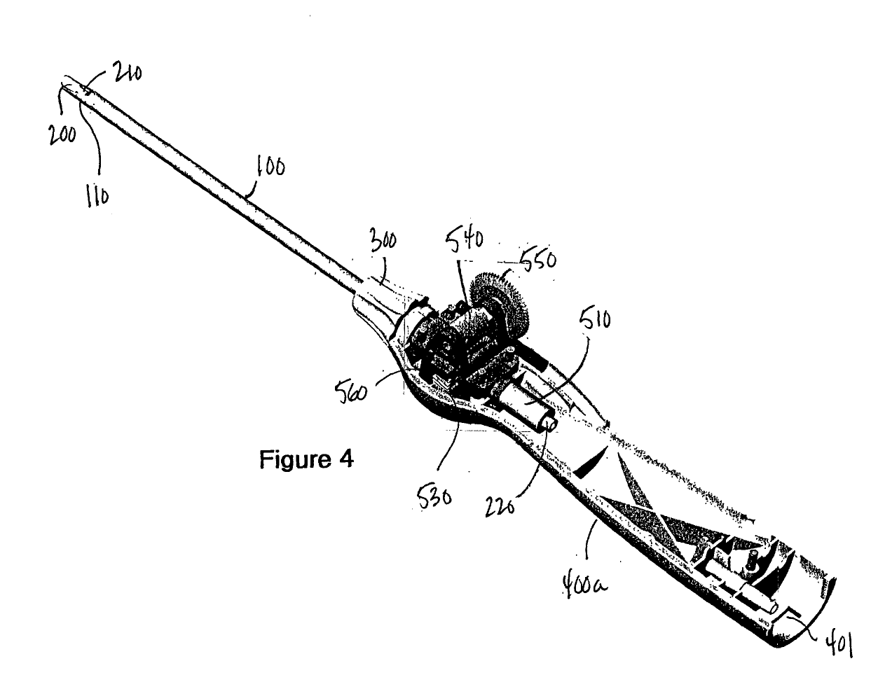

Referring again to FIG. 4, the gear 530 of this embodiment is driven by a worm gear 540 that is, in turn, driven by a rotor 550, and all of these components are held by a connector block 560, which also holds distal and proximal support tubes 500, 510. The connector block 560 is attached to the body 400. In operation, the rotor 550 drives the worm gear 540 which, in turn, drives the gear 530. The gear 530 rotates the shaft 220, and the shaft 220 rotates the burr 200.

In various embodiments, the connection between any motor and the shaft 220 may be reversible and/or may also be indirect, such as, for example, through a belt, a shaft, a hose, or one or more gears. In some embodiments, the motor may be partially or wholly external to the body 400 as, for example, where the motor is a fluid-driven motor in fluid communication with a source of pressurized fluid delivered to a turbine or rotor within the body or is an externally positioned electric motor drivingly coupled via a flexible drive shaft or other suitable means to the shaft of the instrument. In some alternative embodiments (not shown), the rotatable shaft may be inserted into a collet, a chuck, or a similar device, which is itself directly or indirectly driven by a motor. The collet, chuck, or similar device may be mounted within the body or may be on the outside of the body. In still other embodiments, rotation of the shaft and, in turn, the burr, may be effected without a motor, for example, by hand.

Burr rotation speeds achievable by instruments provided according to certain embodiments of the invention are not limited, but may be at least 5,000 rpm, at least about 10,000 rpm, or at least about 20,000 rpm. With suitable motors (e.g., certain liquid-jet driven rotor motors), speeds of at least 30,000 rpm, or at least 50,000 rpm, or of over 100,000 rpm, can be obtained with some embodiments of the invention.

FIG. 6a shows an embodiment of the invention that employs stand-offs. The burr 200, which in the illustrated embodiment has a diameter larger than that of the shaft 220, is mounted on the shaft 220, which may be either solid (as illustrated) or hollow, as described above. The shaft 220 is driven by a motor, such as a turbine, a liquid-jet driven rotor motors, or an electric motor, that is located in the body 400. The shaft 220 is supported by two support members 520 that are also located in the body 400. As in other embodiments, the shaft 220 is surrounded by a sheath 100. The sheath 100 of this embodiment extends to the end of the burr 200 on the top side 140a, but is tapered so as to reveal a portion of the burr 200 on the bottom side 140b.

The inner surface of the sheath 100 of this embodiment is provided with standoffs 700, which may be of any suitable design and are constructed to tolerate intermittent contact with the rotating shaft 220 during operation upon application of sufficient lateral force to the burr 200 and shaft 220. The burr 200 has a minimum separation from the surrounding sheath 100 that is greater than the minimum separation between the stand-offs 700 and the shaft 220. As such, upon lateral deflection of the shaft 220 relative to the sheath 100, contact of the burr 200 with the sheath 100 will be avoided because the relative lateral movement will be arrested when the minimum separation at the stand-offs 700 drops to zero. This will occur before the burr 200 contacts the sheath 100.

Stand-offs 700 do not contact both the sheath 100 and the shaft 200 during normal operation of the instrument; rather, in the absence of lateral forces, there is a minimum separation between the shaft 220 and the stand-offs 700 (where the standoff 700 is adjacent to or a part of the sheath 100, as illustrated), between the sheath 100 and the stand-off 700 (where the stand-off 700 is adjacent to or a part of the shaft 220), or, in alternative embodiments (not shown), between two stand-offs 700 (where one stand-off 700 is adjacent to or a part of the shaft 220 and one stand-off 700 is adjacent to or part of the sheath 100). Stand-offs 700 can, in some embodiments, be shaped and arranged so that the amount of deflection of the shaft 220 possible in all radial directions is approximately the same (and, in any event, is less in all directions than the amount of deflection sufficient for the burr 200 to contact the sheath 100). This may be accomplished, in certain embodiments, by the use of one or more annular stand-offs (e.g., annular ribs) or by the use of individual stand-offs that may be discrete structures uniformly spaced around the inside of the sheath, or, alternatively, by the use of longitudinal ribs, as shown in FIG. 7.

Referring again to FIG. 6, stand-offs 700 may be positioned at any suitable point along the length of the shaft 220 and, in some embodiments, more than one stand-off, of the same or different types, may be used. In some embodiments, it may be advantageous to position the stand-off(s) 700 in a distal region of the shaft, because, due bending along the length of the shaft 220, stand-off(s) 700 at such a position may be more effective at arresting relative deflection of the shaft 220 than would be a stand-off(s) at a more proximal position. In other words, because the displacement of a bending shaft relative to its original axis may be greater at its distal end than at its proximal end, stand-off(s) positioned near the distal end of the shaft 220 may contact the bending shaft 220 earlier than would the same stand-off(s) at a more proximal position. In one embodiment, as illustrated, stand-offs 700 are positioned at a single longitudinal position just proximal of the burr 200.

In some embodiments employing stand-offs, it may be desirable to facilitate the flow of fluid through the sheath 220 in the space between the shaft 220 and the sheath 100. In such cases, stand-offs in the form of distinct knobs, pins, longitudinal ribs, or the like are advantageously used, or, alternatively, annular stand-offs that have one or more openings (holes, slots, notches, etc.) may be employed, to allow for improved fluid flow in the axial direction as compared to annular stand-off(s) without such openings. In the embodiment illustrated in FIG. 6b, for example, the standoffs 700 comprise discontinuous annular ribs that are configured to allow fluid and debris to flow from the area of the burr 200, through the channel 230 formed between the shaft 220 and the sheath 100, and through the tube 410. Because the debris is generally small in diameter compared to the channel 230, only a small amount of suction is typically required to remove the debris from the area of the burr 200. Sufficient suction can be provided, in some embodiments, by elevation of a bag of saline (not shown) used for irrigating the surgical site. The flow of fluid away from the surgical site may also be facilitated by the design of the burr 200, as previously described.

In some embodiments, the outer surface of the shaft 220, the inner surface of the sheath 100, and/or various surfaces of the stand-offs 700 are provided with a relatively smooth finish, so as to prevent damage upon any of these surfaces coming into contact with each other. In some embodiments, these surfaces may covered with a protective and/or lubricious coating to minimize friction.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, provided that such features, systems, materials and/or methods are not mutually inconsistent, is included within the scope of the present invention. In the claims, all transitional phrases or phrases of inclusion, such as “comprising,” “including,” “carrying,” “having,” “containing,” “composed of,” “made of,” “formed of” and the like are to be understood to be open-ended, i.e. to mean “including but not limited to.” Only the transitional phrases or phrases of inclusion “consisting of” and “consisting essentially of” are to be interpreted as closed or semi-closed phrases, respectively, as set forth in MPEP section 2111.03.

What is claimed is:

1. A rotatable component-providing surgical instrument, comprising:
a body having a distal end and a proximal end;
a shaft rotatably supported by the body and extending from the distal end of the body;
a tissue contacting component drivable by the shaft; and
an outer tube connected to the distal end of the body and surrounding at least a portion of the shaft, wherein a minimum separation between the tissue contacting component and the outer tube is greater than a minimum separation between the shaft and the outer tube.

2. The surgical instrument of claim 1, wherein a maximum radial diameter of the tissue contacting component is smaller than a minimum radial diameter of the shaft.

3. The surgical instrument of claim 1, wherein a maximum radial diameter of the tissue contacting component is approximately the same as a maximum radial diameter of the shaft.

4. The surgical instrument of claim 1, wherein an interior diameter of the outer tube in a region adjacent to the shaft is smaller than an interior diameter of the outer tube in a region adjacent to the tissue contacting component.

5. The surgical instrument of claim 1, wherein the distal portion of the shaft is free of contact with any support elements.

6. The surgical instrument of claim 1, wherein the outer tube is flexibly connected to the body.

7. The surgical instrument of claim 1, further comprising a flexible connector positioned at least in part between the body and the outer tube.

8. The surgical instrument of claim 8, wherein flexible connector is a resilient boot.

9. The surgical instrument of claim 1, wherein the outer tube is constructed of a rigid material.

10. The surgical instrument of claim 1, wherein the outer tube surrounds at least a portion of the tissue contacting component.

11. The surgical instrument of claim 11, wherein the portion of the tissue contacting component surrounded by the outer tube is variable.

12. The surgical instrument of claim 1, wherein the shaft has a longitudinal axis and the outer tube is constructed and arranged to be movable in the direction of the axis.

13. The surgical instrument of claim 1, wherein the outer tube further comprises a radiopaque marker.

14. The surgical instrument of claim 1, wherein the shaft further comprises a lumen adapted for fluid flow therein.

15. The surgical instrument of claim 1, further comprising a motor coupled to the shaft.

16. The surgical instrument of claim 15, wherein the motor comprises a liquid jet-driven rotatable rotor.

17. The surgical instrument of claim 1, wherein the resistance to deflection of the outer tube to a laterally applied force at a given longitudinal point is at least 10 percent less than the resistance to deflection of the shaft to the same force at the same longitudinal point.

18. The surgical instrument of claim 17, wherein the resistance to deflection of the outer tube to a laterally applied force at a given longitudinal point is at least 50 percent less than the resistance to deflection of the shaft to the same force at the same longitudinal point.

19. The surgical instrument of claim 18, wherein the resistance to deflection of the outer tube to a laterally applied force at a given longitudinal point is at least 90 percent less than the resistance to deflection of the shaft to the same force at the same longitudinal point.

20. The surgical instrument of claim 1, wherein the outer tube is flexible.

21. The surgical instrument of claim 20, wherein the outer tube is more flexible than the shaft with respect to the body.

22. The surgical instrument of claim 20, wherein the flexibility of the outer tube at a first longitudinal point is different than the flexibility of the outer tube at a second longitudinal point.

23. The surgical instrument of claim 1, further comprising an evacuation channel within the outer tube.

24. The surgical instrument of claim 1, further comprising an irrigation channel within the outer tube.

25. The surgical instrument of claim 1, further comprising at least one stand-off positioned between the outer tube and the shaft.

26. The surgical instrument of claim 25, wherein the at least one stand-off is constructed and arranged to allow fluid to flow through the outer tube.

27. The surgical instrument of claim 25, wherein the at least one stand-off is positioned proximally adjacent to the tissue contacting component.

28. The surgical instrument of claim 1, wherein the outer tube is constructed and arranged such that, upon application of lateral force to the tissue contacting component, the outer tube contacts the shaft before contacting the tissue contacting component

29. The surgical instrument of claim 1, wherein the outer tube is flexibly connected to the distal end of the body, thereby rendering the outer flexible with respect to the body, and wherein the instrument further comprises at least one standoff positioned between the shaft and the outer tube.

30. The surgical instrument of claim 1, further comprising:
a flexible connector member positioned on the distal end of the body flexibly connecting the outer tube to the distal end of the body, thereby rendering the outer tube more flexible than the shaft with respect to the body, and wherein the outer tube is constructed and arranged such that, upon application of lateral force to the tissue contacting component, the outer tube contacts the shaft before contacting the tissue contacting component.

31. The surgical instrument of claim 30, further comprising at least one stand-off positioned between the shaft and the outer tube.

32. A rotatable component-providing surgical instrument, comprising:
a body having a distal end and a proximal end;
a shaft rotatably supported by the body and extending from the distal end of the body;
a tissue contacting component drivable by the shaft;
an outer tube surrounding at least a portion of the shaft, wherein the outer tube is constructed and arranged such that, upon application of lateral force to the tissue contacting component, the outer tube contacts the shaft before contacting the tissue contacting component.

33. The surgical instrument of claim 32, wherein a maximum radial diameter of the tissue contacting component is smaller than a minimum radial diameter of the shaft.

34. The surgical instrument of claim 32, wherein a maximum radial diameter of the tissue contacting component is approximately the same as a maximum radial diameter of the shaft.

35. The surgical instrument of claim 32, wherein an interior diameter of the outer tube in a region adjacent to the shaft is smaller than an interior diameter of the outer tube in a region adjacent to the tissue contacting component.

36. The surgical instrument of claim 32, wherein the distal portion of the shaft is free of contact with any support elements.

37. The surgical instrument of claim 32, wherein the outer tube surrounds at least a portion of the tissue contacting component.

38. The surgical instrument of claim 32, further comprising a motor coupled to the shaft.

39. The surgical instrument of claim 32, wherein the outer tube is more flexible than the shaft with respect to the body.

40. A rotatable component-providing surgical instrument, comprising:
a body having a distal end and a proximal end;
a shaft rotatably supported by the body and extending from the distal end of the body;
a tissue contacting component drivable by the shaft;
an outer tube flexibly connected to the distal end of the body and surrounding at least a portion of the shaft; and
at least one stand-off constructed and arranged such that, upon application of a lateral force to the tissue contacting component, the at least one stand-off contacts the shaft before the tissue contacting component contacts the outer tube.

41. The surgical instrument of claim 40, further comprising a flexible connector positioned at least in part between the body and the outer tube.

42. The surgical instrument of claim 41, wherein the flexible connector is a resilient boot.

43. The surgical instrument of claim 40, wherein the outer tube surrounds at least a portion of the tissue contacting component.

44. The surgical instrument of claim 40, further comprising a motor coupled to the shaft.

45. The surgical instrument of claim 40, wherein the at least one stand-off is constructed and arranged to allow fluid to flow through the outer tube.

46. The surgical instrument of claim 40, wherein the at least one stand-off is positioned proximally adjacent to the tissue contacting component.

47. A rotatable component-providing surgical instrument, comprising:
a body having a distal end and a proximal end;
a shaft rotatably supported by the body and extending from the distal end of the body;
a tissue contacting component drivable by the shaft;
a flexible connector member positioned at the distal end of the body; and
an outer tube connected to the flexible connector member and surrounding at least a portion of the shaft.

48. The surgical instrument of claim 47, wherein a maximum diameter of the tissue contacting component is smaller than a minimum diameter of the shaft.

49. The surgical instrument of claim 47, wherein a maximum diameter of the tissue contacting component is approximately the same as a maximum diameter of the shaft.

50. The surgical instrument of claim 47, wherein an interior diameter of the outer tube in a region adjacent to the shaft is smaller than an interior diameter of the outer tube in a region adjacent to the tissue contacting component.

51. The surgical instrument of claim 47, wherein the distal portion of the shaft is free of contact with any support elements.

52. The surgical instrument of claim 47, wherein flexible connector is a resilient boot.

53. The surgical instrument of claim 47, wherein the outer tube surrounds at least a portion of the tissue contacting component.

54. The surgical instrument of claim 47, further comprising a motor coupled to the shaft.

55. A rotatable component-providing surgical instrument, comprising:
a body having a distal end and a proximal end;
a shaft rotatably supported by the body and extending from the distal end of the body;
a tissue contacting component drivable by the shaft; and
an outer tube connected to the distal end of the body and surrounding at least a portion of the shaft, wherein the outer tube is more flexible than the shaft with respect to the body.

56. The surgical instrument of claim 55, wherein the distal portion of the shaft is free of contact with any support elements.

57. The surgical instrument of claim 55, wherein the outer tube is constructed of a rigid material.

58. The surgical instrument of claim 57, wherein the outer tube is made of stainless steel.

59. The surgical instrument of claim 55, wherein the outer tube surrounds at least a portion of the tissue contacting component.

60. The surgical instrument of claim 55, further comprising a motor coupled to the shaft.

61. The surgical instrument of claim 55, wherein the resistance to deflection of the outer tube to a laterally applied force at a given longitudinal point is at least 10 percent less than the resistance to deflection of the shaft to the same force at the same longitudinal point.

62. The surgical instrument of claim 55, wherein the resistance to deflection of the outer tube to a laterally applied force at a given longitudinal point is at least 50 percent less than the resistance to deflection of the shaft to the same force at the same longitudinal point.

63. The surgical instrument of claim 55, wherein the resistance to deflection of the outer tube to a laterally applied force at a given longitudinal point is at least 90 percent less than the resistance to deflection of the shaft to the same force at the same longitudinal point.

64. A shaft assembly for use in a surgical instrument, comprising:
a rotatable shaft;
a tissue contacting component drivable by the shaft; and
an outer tube surrounding at least a portion of the shaft, wherein a minimum separation between the tissue contacting component and the outer tube is greater than a minimum separation between the shaft and the outer tube.

65. The shaft assembly of claim 64, wherein the distal portion of the shaft is free of contact with any support elements.

66. The shaft assembly of claim 64, wherein the outer tube surrounds at least a portion of the tissue contacting component.

67. The shaft assembly of claim 64, further comprising at least one stand-off positioned between the outer tube and the shaft.

68. A method comprising:
providing a surgical instrument including a body having a distal end and a proximal end, a shaft roatably supported by the body and extending from the distal end of the body, a tissue contacting component drivable by the shaft, and an outer tube positioned to surround at least a portion of the shaft and tissue contacting component and having an inner surface normally radially spaced from an outer surface of the shaft;
contacting the tissue contacting component with tissue of a patient;
applying a force to the tissue via the shaft and tissue contacting component, at least a portion the force being laterally directed with respect to the shaft; and
laterally displacing at least a portion of the shaft with respect to the outer tube in response to application of the force, thereby decreasing a radial spacing between the outer surface of the shaft and the inner surface of the outer tube without contact between the inner surface of the outer tube and the tissue contacting component.

69. The method of claim 69, further comprising the step of contacting the outer surface of the shaft with the inner surface of the outer tube, upon application of sufficient lateral force to the shaft, while maintaining essentially free of contact the tissue contacting component and the inner surface of the outer tube.

70. A method comprising:
providing a surgical instrument including a body having a distal end and a proximal end, a shaft roatably supported by the body and extending from the distal end of the body, a tissue contacting component drivable by the shaft, an outer tube positioned to surround at least a portion of the shaft and tissue contacting component and having an inner surface normally radially spaced from an outer surface of the shaft, and at least one stand off positioned between the shaft and the outer tube;
contacting the tissue contacting component with tissue of a patient;
applying a force to the tissue via the shaft and tissue contacting component, at least a portion the force being laterally directed with respect to the shaft; and
laterally displacing at least a portion of the shaft with respect to the outer tube in response to application of the force, thereby decreasing a radial spacing between the outer surface of the shaft and the inner surface of the outer tube without contact between the inner surface of the outer tube and the tissue contacting component

71. The method of claim 71, further comprising the step of contacting the outer surface of the shaft with at least one stand off, upon application of sufficient lateral force to the shaft, while maintaining essentially free of contact the tissue contacting component and the inner surface of the outer tube.
